(19) United States
(12) Patent Application Publication
(10) Pub. No.: US 2005/0019410 A1
(43) Pub. Date: Jan. 27, 2005

(54) Preparation of microparticles

(76) Inventor: Richard Alan Johnson, Nottingham (GB)

Correspondence Address: Edwin V.Merkel;Nixon Peabody, Clinton Square, PO Box 31051, Rochester, NY, 14603-1051, US

(21) Appl. No.: 10/486,633

(22) PCT Filed: Jul. 31, 2002
(86) PCT No.: PCT/GB02/03491

(30) Foreign Application Priority Data
Aug. 17, 2001 (GB) 0120123.5

(51) Int. Cl. A61K 9/14; B29B 9/00

(52) U.S. Cl. 424/489; 264/5

(57) ABSTRACT

A method for producing microparticles of a particle-forming material comprises the steps of: a) forming a suspension of the particle-forming material; and b) spray-drying said suspension. The formation of a suspension of the particle-forming material is preferably carried out by first dissolving the particle-forming material in a solvent, and then adding to the solution so formed a non-solvent for the particle-forming material, so as to bring about precipitation of the particle-forming material. The microparticles produced in accordance with the invention may be useful in therapeutic applications or in diagnostic imaging.

This invention relates to the preparation of microparticles, and in particular to the preparation of proteinaceous material in the form of fine particles.

The preparation of microparticles of proteinaceous materials such as albumin is well-documented. A technique that has been used is spray-drying, in which a solution of the proteinaceous material in a solvent, most commonly water, is sprayed into a heated gas-filled chamber such that the solvent evaporates to form microparticles which are then separated from the gas.

There is continued interest in developing new and improved techniques for making microparticles containing both drugs and imaging agents, and this involves numerous challenges. For instance, there is a need to control the particle size distribution closely. For products intended for intravenous administration, it is desirable to make particles less than 6 μm in size. Similarly, for products intended for delivery by inhalation to the lung it is usually beneficial to produce microparticles with an aerodynamic size suitable for penetration deep into the lung. Particles for nasal administration may be rather larger, eg with a particle size of several tens of μm.

Similarly, there may be a need to encapsulate or otherwise incorporate therapeutic agents (drugs) or contrast agents. By encapsulating drugs and contrast agents it is hoped that they will remain in the circulation for longer, enhancing performance and possibly reducing toxicity.

As well as the spray-drying techniques noted above, a number of other methods have been used to make microparticles smaller than 10 μm, for both imaging and therapeutic purposes. These methods include emulsification, precipitation and milling. However, all these existing methods still have a number of disadvantages:
(i) Presence of significant amounts of particles over 6 μm in size. This is important since if administered intravenously these larger particles will block capillaries etc, and if administered to the lung will fail to penetrate deep into the airways.

(ii) A requirement for the incorporation of surfactants to avoid agglomeration and to maintain a relatively homogeneous suspension—the presence of such additional ingredients, which are of no therapeutic or functional benefit, and which could be harmful, is generally undesirable.

(iii) The products obtained in some emulsion-based processes may be relatively hydrophobic, and consequently difficult to disperse.

There has now been devised an improvement to known spray-drying processes for the preparation of microparticles, that overcomes or substantially mitigates the above-mentioned and/or other disadvantages of the prior art.

According to the invention, there is provided a method for producing microparticles of a particle-forming material, which method comprises the steps of
a) forming a suspension of the particle-forming material; and
b) spray-drying said suspension.

Step a), ie the formation of a suspension of the particle-forming material, is preferably carried out by first dissolving the particle-forming material in a solvent, and then adding to the solution so formed a non-solvent for the particle-forming material, so as to bring about precipitation of the particle-forming material. By a “non-solvent” is meant a liquid in which the solubility of the particle-forming material is substantially less than the solubility of the particle-forming material in the solvent, but which is miscible with the solvent.

The non-solvent is preferably added in excess, ie the volume of non-solvent added to the solvent is preferably greater than the volume of the solution of the particle-forming material in the solvent. In other words, the solvent/non-solvent mixture that is spray-dried in step b) most preferably comprises in excess of 50% v/v of non-solvent, more preferably in excess of 60% v/v, and possibly in excess of 70% v/v.

Most commonly, the solvent is water. The preferred non-solvent is ethanol. In general, however, any suitable combination of solvent and non-solvent may be used, provided that the addition of non-solvent has the desired effect of causing precipitation of the particle-forming material and that the solvent and the non-solvent are miscible in the proportions used.

Although the solvent is most commonly water, it may alternatively be, for example, an organic solvent. In such a case, the non-solvent may be water, and the use of the non-solvent may then be beneficial in reducing risks associated with the subsequent spray-drying of the suspension containing the possibly flammable organic solvent.

Where the particle-forming material is, as is preferred, a proteinaceous material, the precipitation by addition of the non-solvent is preferably carried out at a pH which is removed from the isoelectric point, so as to prevent or minimise agglomeration of the suspended particles and to produce hydrophilic particles that are readily susceptible to dispersion after spray-drying. In this way, the use of additional surfactants to achieve the same objectives may be avoided. The same may be true in the case of non-proteinaceous particle-forming materials, if those materials too exhibit an isoelectric point.

Step b), ie spray-drying of the suspension formed in step a), may be carried out in a generally conventional manner, using equipment of a generally conventional nature. In outline, the spray-drying process involves spraying the suspension into a chamber containing a heated gas, most commonly air. This causes the solvent/non-solvent mixture to evaporate and produces solid microparticles. The gas is drawn from the chamber, and the microparticles entrained in the gas are separated from the gas, eg by means of cyclonic separator or some form of filter arrangement. The microparticles are then collected in a suitable receptacle.

The properties of the microparticles obtained by the spray-drying process are dependent on a number of factors. These include the flow rate of gas through the spray-drying apparatus, the concentration of the particle-forming material in the suspension, the nature of the solvent and non-solvent, the rate at which the suspension is fed into the spray-drying apparatus and the temperature of the gas in the chamber. Usually, small size distributions can be achieved by a combination of a low suspension feed rate, a high degree of atomization and high flow rate of gas.

It is particularly preferred that, between step a) and step b), ie after formation of the suspension but before spray-drying, the suspension is subjected to homogenisation, eg by mechanical agitation. This results in a more even size distribution of particles in the suspension, and a correspondingly smaller and more even size distribution of the microparticles formed in step b).

The particle-forming material is most preferably proteinaceous, which includes non-naturally occurring polypeptides and polyamino acids. For example, the particle-forming material may be collagen, gelatin or albumin. Albumin is a particularly preferred material. Where the microparticles are intended for administration to the human body, the particle-forming material is preferably of human origin, ie actually derived from humans or identical (or substantially so) in structure to protein of human origin. A particularly preferred particle-forming material is thus human serum albumin which may, for instance, be obtained from donated blood or may be derived from the fermentation of microorganisms (including cell lines) that have been transformed or transfected to express human serum albumin. Non-proteinaceous materials that may be used as particle-forming materials include sugars, carbohydrates, drugs and materials useful as imaging contrast agents.

The suspension preferably contains from 0.1 to 50% w/v of particle-forming material, more preferably 1 to 20% w/v, and most preferably 2 to 10% w/v, particularly when the particle-forming material is albumin. Mixtures of particle-forming materials may be used, in which case the above figures represent the total content of particle-forming material(s).

The method according to the invention is advantageous primarily in that the two stages of the process (formation of a suspension and spray-drying) may be optimised separately to achieve the desired form and size distribution of microparticle. This gives a high degree of control over the properties of the microparticles. In particular, the process enables the production of microparticles with particularly small sizes and particularly narrow size distributions. Microparticles produced in accordance with the invention may, for example, have particle sizes of predominantly less than 4 μm, and number sizes with modal peaks below 1 μm and mean sizes (as measured using a Coulter counter) less than 2 μm. Microparticles with such small sizes are beneficial in that they may enter very small blood vessels (capillaries) and/or may penetrate deep into the lungs. It may also be possible to produce microparticles of larger size, eg for nasal administration.

It will be appreciated that references to the “size” of the microparticles will normally mean the “diameter” of the microparticles, since the microparticles will most commonly be substantially spherical. However, it will also be appreciated that the microparticles may not be spherical, in which case the size may be interpreted as the diameter of a notional spherical particle having a mass equal to that of the non-spherical microparticle.

Furthermore, microparticles produced by spray-drying a suspension in accordance with the invention tend to be solid rather than the hollow microparticles that are typically produced when a solution is spray-dried.

In addition to the advantages noted above, the microparticles produced in accordance with the invention may be devoid of potentially undesirable excipients such as surfactants. Protein microparticles produced by spray-drying a suspension appear to suspend more readily in aqueous suspension without the need for surfactants in comparison with microparticles produced by other methods.

Microparticles produced by spray-drying a suspension are mis-shapen. This may change their flow properties and deaggregation properties compared to round/spherical microparticles that are produced by spray-drying a solution.

The precipitated particles produced in suspension can be coated with an agent to change their solubilisation properties. For example, cholesterol can be added to an Iopamidol suspension in propan-2-ol/chloroform. The cholesterol will dissolve in the solvent mixture. On spray-drying the Iopamidol suspension, the aqueous dissolution properties of the Iopamidol microparticles will be changed. The cholesterol will coat the Iopamidol microparticles, reducing their aqueous solubility.

If the solution contains two or more compounds that are not soluble in the “non-solvent”, when the non-solvent is added a precipitate will form from both compounds. If the suspension is then spray-dried it will produce “mixed” microparticles containing both compounds.

The microparticles produced in accordance with the invention may be useful in therapeutic applications, eg vehicles for the delivery of medicaments, or in diagnostic imaging, eg for imaging techniques using ultrasound, magnetic resonance etc. The particle-forming material may therefore be a therapeutically active agent (ie a drug) or a pharmaceutical excipient, eg cholesterol, or it may be a contrast-enhancing agent for use in diagnostic imaging. Examples of contrast-enhancing agents are X-ray contrast agents, eg Iopamidol, nuclear imaging agents, eg technetium, and magnetic resonance contrast agents. Therapeutically active agents may be incorporated into the microparticles, eg by absorption into and/or adsorption onto or covalent binding to the surface of the microparticles. Alternatively, the microparticles may be formed wholly or in part from the therapeutically active agent(s), ie the therapeutically active agent(s) may constitute, or be part of, the particle-forming material.

The invention will now be illustrated in greater detail, by way of illustration only, with reference to the following Examples and Figures, in which

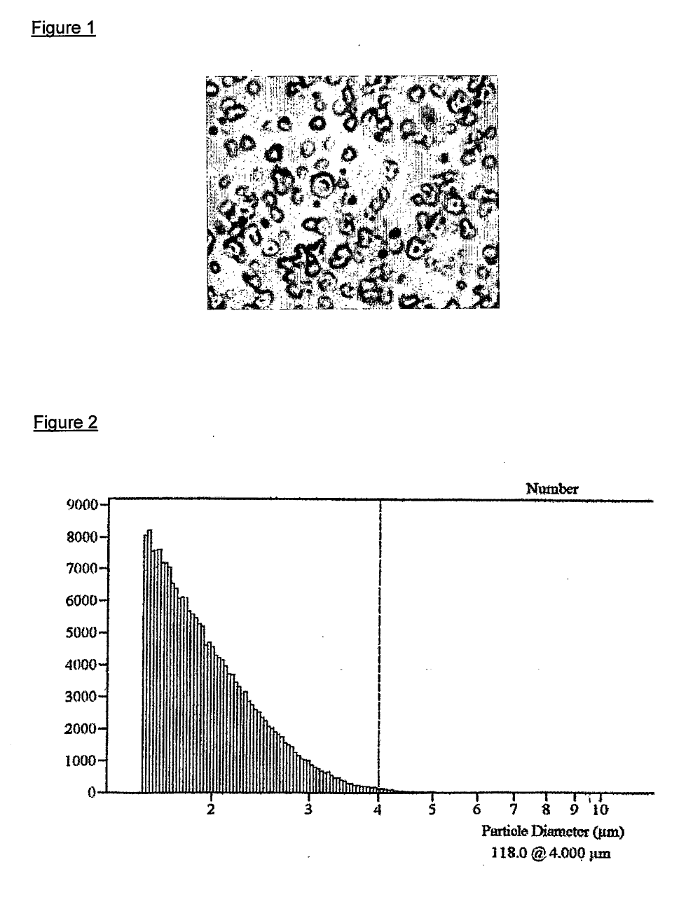

FIG. 1 is a light micrograph (×1000) showing morphology of microparticles produced when precipitated human albumin is spray-dried;

FIG. 2 is a Coulter Size Distribution of albumin microparticles produced by spray-drying a suspension;

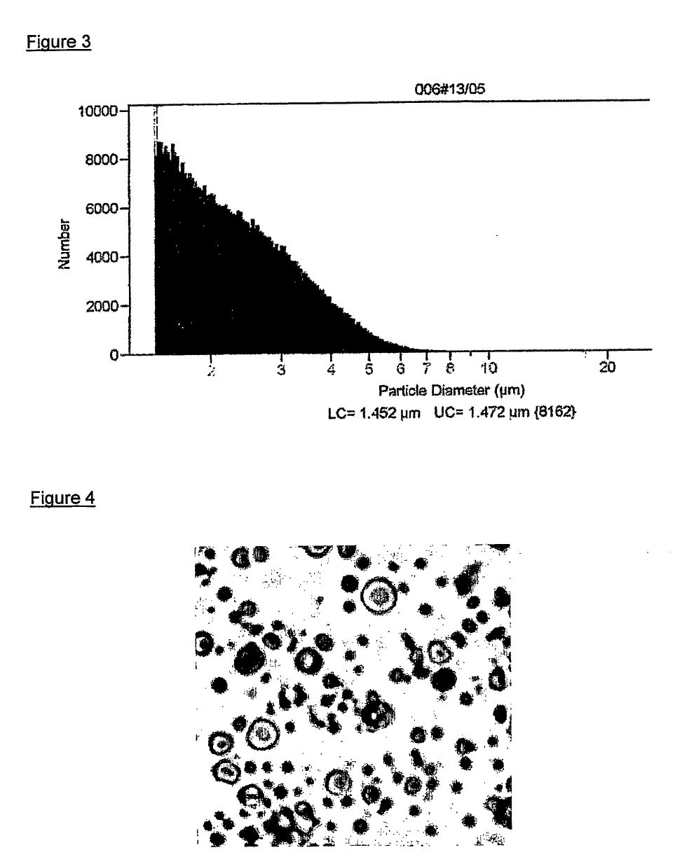

FIG. 3 is a Coulter Size Distribution of albumin microparticles produced by spray-drying a solution;

FIG. 4 is a light micrograph (×1000) showing morphology of microparticles produced when soluble human albumin is spray-dried;

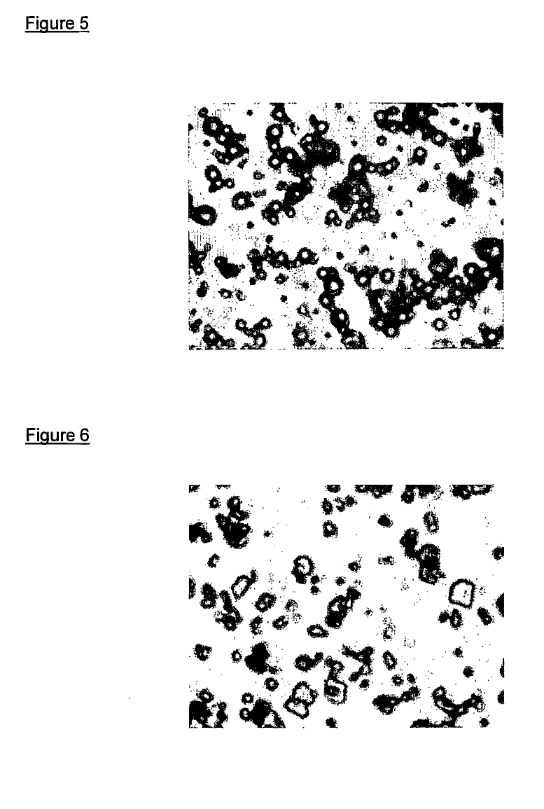

FIG. 5 is a light micrograph (×1000) showing morphology of microparticles produced when cholesterol solution is spray-dried;

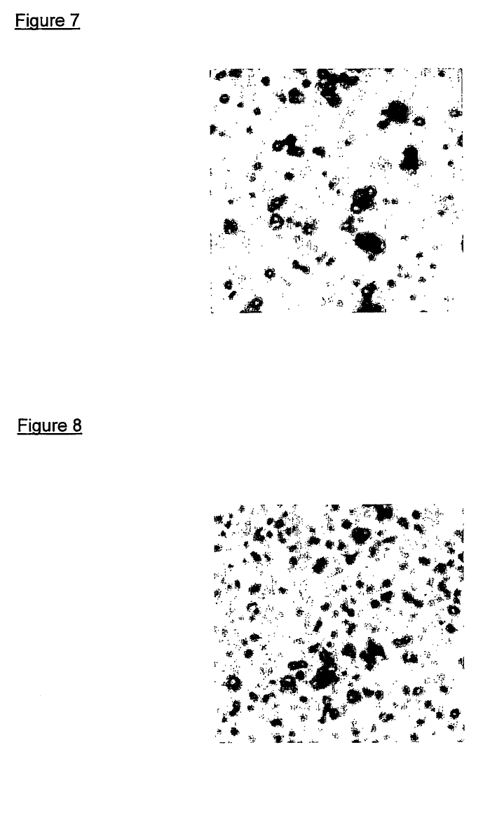

FIG. 6 is a light micrograph (×1000) showing morphology of microparticles produced when cholesterol suspension is spray-dried; FIG. 7 is a light micrograph (×1000) showing morphology of microparticles produced when Iopamidol solution is spray-dried; and

FIG. 8 is a light micrograph (×1000) showing morphology of microparticles produced when Iopamidol suspension is spray-dried;

### EXAMPLE 1

Spray-Drying of Precipitated Human Albumin

Method

A 100 ml volume solution of Human Serum Albumin (20% w/v, USP grade) was dialysed against 2000 ml volumes of pyrogen-free purified water (PFPW) overnight at room temperature to remove excess sodium chloride.

The following solution was prepared, with all work being done at 24° C., and the ethanol being added slowly, with gentle homogenisation with a Janke and Kunkel T25 homogeniser (8000 rpm) whilst adding the ethanol:

| | |
|---|---|
| Dialysed Human Serum Albumin (12% w/v), pH 7.0 | 40 ml |
| Ethanol | 60 ml |

It was noted that the straw-coloured solution changed to a milky-white suspension when the ethanol was added.

The suspension was further homogenised using a Janke & Kunkel T25 homogeniser at 20,500 rpm for 30 seconds.

The homogenised suspension was spray-dried at the following settings using a Buchi Mini Spray Dryer model B-191, fitted with a Schlick 2-fluid atomisation nozzle (model 970/0). The following spray-drying conditions were used:

| | | |
|---|---|---|
| Inlet temperature | 100° | C. |
| Starting outlet temperature | 67° | C. |
| Liquid feed rate | 3 | ml/min |
| Atomisation pressure | 4.0 | barg |
| Drying air setting | 100% | |

The microparticles were recovered from the cyclone collection jar.

To enable aqueous sizing on a Coulter counter, a 200 mg sample of the microcapsules were rendered insoluble by heating in a Gallenkamp laboratory oven at 175° C. for 60 minutes.

The insoluble microparticles were de-agglomerated (to break up aggregates) using a Fritsch Pulverisette 14 centrifugal mill running at maximum speed with a 12-tooth rotor and 0.5 mm screen.

To achieve de-agglomeration the 200 mg aliquot of heat fixed microcapsules was mixed with 600 mg of anhydrous mannitol and blended with a spatula. The mixture was fed into the mill over a 10 second period whilst the mill was operating at maximum speed.

Light Micrography

A small quantity (approx 100 mg) of the deagglomerated microparticles were suspended in 5 ml of water. Light microscopy was used to determine the particle morphology in the aqueous suspension. A small drop of suspension was placed on a microscope slide and a cover slip applied. An image at ×1000 magnification was obtained using a Nikon Labophot microscope.

Images were captured using a SeeScan image analyser. The captured images were printed using a Sony Mavigraph colour video printer.

Sizing

Prior to sizing, 50 mg of the de-agglomerated microcapsules were re-suspended in 1 ml of PFPW and vortexed gently. The microcapsules were sized in Isoton on a Coulter Multisizer II fitted with a 70 μm orifice tube.

Results

The de-agglomerated microcapsules exhibited excellent re-suspension properties in the PFPW. No surfactant addition was necessary to achieve good re-suspension.

The microparticles were found to have a small mean size. (number distribution) of 1.97 μm and the following size distribution (by number, measured as described above):

| % greater than | |
|---|---|
| 3 μm | 7.51% |
| 4 μm | 0.92% |
| 5 μm | 0.13% |
| 6 μm | 0.03% |

The images obtained by light micrography (see FIG. 1) confirmed that the microparticles produced by spray-drying of precipitated albumin were somewhat mishapen. Closer analysis confirmed that the majority of the microparticles were not hollow in appearance.

The Coulter size distribution is shown in FIG. 2.

### EXAMPLE 2

Spray-Drying of Human Albumin as a Solution (Comparative Example)

Method

In contrast to spray-drying a precipitated suspension (Example 1), human albumin was spray-dried as a solution.

A 250 ml volume of human albumin (USP Grade, supplied as 20% w/v by Grifols, Spain) was dialysed against 5.0 litres of purified water. The albumin was sealed in dialysis tubing and the water stirred overnight at room temperature.

The albumin concentration was determined by absorbance at 280 nm (assume an extinction coefficient of 0.53 for a 1.0 mg/ml solution).

The following solution was prepared for spray-drying:
Human Albumin 4.8% (w/v)* in water, pH 7.0.
(* note that this is the same albumin concentration as in Example 1)

The albumin solution was spray-dried under the same conditions as in Example 1

The microparticles were collected and heat-insolubilised as described in Example 1.

For further analyses, the heat-insolublised microparticles were sonicated in ethanol to ensure that any agglomerates were disrupted. On subsequent aqueous suspension the microparticles were seen to be dispersed with no significant agglomeration (determined by light microscopy)

The microparticles were sized using a Coulter Multisizer as described above (Example 1)

The microparticles were subjected to light microscopy (×1000 magnification) as described in Example 1.

Results

Coulter size analysis revealed that the microparticles had the following sizes:

| % greater than | |
|---|---|
| 3 μm | 22.69% |
| 4 μm | 7.15% |
| 5 μm | 1.80% |
| 6 μm | 0.39% |

The size data (see FIG. 3) confirmed that the microparticles produced by spray-drying an albumin solution were significantly larger than those produced by spray-drying a precipitated suspension of the same albumin concentration (Example 1).

Closer (light microscopic) analysis revealed that the microparticles produced by spray-drying an albumin solution were predominantly spherical in shape and predominantly hollow. Intact microparticles contained air and appeared as “black” bubbles under the microscope. In contrast the microparticles produced by spray-drying a suspension were somewhat mishapen. They were not hollow/air-containing.

A typical light micrograph is shown in FIG. 4.

### EXAMPLE 3

Spray-Drying of Cholesterol as a Suspension, Compared to a Solution

Cholesterol is a pharmaceutical excipient that can be used to formulate a variety of drugs. These applications include oral, topical and injectable drug formulations.

Cholesterol is insoluble in water but can be dissolved in organic solvents such as propan-2-ol and chloroform.

A comparative study was undertaken in which cholesterol was spray-dried as a solution (in propan-2-ol) compared to a suspension (precipitated from propan-2-ol by addition of a non-solvent, water).

Method

A 2.5 g quantity of cholesterol (Sigma, 99+%) was dissolved in 125 ml of propan-2-ol.

Spray-Drying as a Solution:

A 50 ml volume of the cholesterol solution was spray-dried using a Buchii spray dryer (see Example 1 for dryer specification).

The following spray-drying conditions were used:

| | | |
|---|---|---|
| Inlet temperature | 50° | C. |
| Outlet temperature | 42° | C. |
| Liquid feed rate | 1.5 | ml/min |
| Atomisation pressure | 1.0 | barg |
| Drying air setting | 100% | |

The cholesterol solution was spray-dried to yield a fine white powder that was collected by the spray dryer cyclone.

Light microscopy was undertaken on the dry powder collected. The microparticles were spread onto the surface of a microscope slide and analysed using a Nikon Labophot microscope (×1000 magnification) as described in Example 1 above. A typical light micrograph is shown in FIG. 5.

Spray-Drying as a Suspension:

The remaining 75 ml of cholesterol solution was placed on a magnetic stirrer and stirred at a medium setting.

The cholesterol was precipitated by addition of a non-solvent. In this example precipitation was achieved by slowly adding 25 mls of purified water (over a period of approx 30 seconds).

The milky white suspension was spray-dried using the same conditions as described for the spray-drying of cholesterol solution. The microparticles produced were collected and analysed by light microscopy as described for the above sample. A typical light micrograph is shown in FIG. 6.

Results

The results obtained confirm that the cholesterol microparticles produced by spray-drying of a solution are predominantly spherical in appearance and appear to be hollow.

In contrast the cholesterol microparticles produced by spray-drying a suspension were mishappen and appeared crystalline in appearance.

### EXAMPLE 4

Spray-Drying of Iopamidol as a Suspension, Compared to a Solution

Iopamidol is an X-ray contrast agent that is used primarily to enhance images of major blood vessels such as the coronary arteries. It is supplied as a sterile solution for injection. It is highly soluble in water.

There are several potential applications in which a dry powder formulation of the contrast agent may be advantageous. For example, dry microparticles of Iopamidol can be further treated to produce an encapsulated formulation that offers the possibilty of targeting the contrast agent to other organs (such as the liver).

A comparative study was undertaken in which Iopamidol was spray-dried as a solution (in propan-2-ol) compared to a suspension (precipitated from propan-2-ol by addition of chloroform).

Method

Spray-Drying as a Solution:

Commercially available Iopamidol was diluted to give a solution for spray-drying:

| | |
|---|---|
| lopamidol* | 10 ml |
| Water | 65 ml |
| Ethanol | 25 ml |

(* Commercially available contrast agent with a concentration of 612 mg Iopamidol/ml)

The solution was spray-dried using a Buchii spray dryer (see Example 1 for dryer specification).

The following spray-drying conditions were used:

| | | |
|---|---|---|
| Inlet temperature | 60° | C. |
| Outlet temperature | 47° | C. |
| Liquid feed rate | 1.5 | ml/min |
| Atomisation pressure | 2.0 | barg |
| Drying air setting | 100% | |

The Iopamidol solution was spray-dried to yield a fine white powder that was collected by the spray dryer cyclone.

Light microscopy was undertaken on the dry powder collected. The microparticles were spread onto the surface of a microscope slide and analysed using a Nikon Labophot microscope (×1000 magnification) as described in Example 1 above. A typical light micrograph is shown in FIG. 7.

Spray-Drying as a Suspension:

Iopamidol was obtained as a dry powder for use in this study. The dry Iopamidol was obtained by spray-drying a commercially available solution of Iopamidol as described above.

A 2.0 g quantity of dry Iopamidol was dissolved in 50 ml of propan-2-ol using a magnetic stirrer and also warming slightly to aid dissolution.

The Iopamidol solution was precipitated by the addition of 50 ml of chloroform (a miscible non-solvent) whilst stirring at medium speed. The chloroform was added over a 30 second period.

The suspension was spray-dried using a Buchii spray dryer (see Example 1 for dryer specification).

The following spray-drying conditions were used:

| | | |
|---|---|---|
| Inlet temperature | 60° | C. |
| Outlet temperature | 47° | C. |
| Liquid feed rate | 1.5 | ml/min |
| Atomisation pressure | 2.0 | barg |
| Drying air setting | 100% | |

The Iopamidol suspension was spray-dried to yield a fine white powder that was collected by the spray dryer cyclone.

Light microscopy was undertaken on the dry powder collected. The microparticles were spread onto the surface of a microscope slide and analysed using a Nikon Labophot microscope (×1000 magnification) as described in Example 1 above. A typical light micrograph is shown in FIG. 8.

Results

The results obtained confirm that the Iopamidol microparticles produced by spray-drying of a solution are predominantly spherical in appearance and appear to be hollow (see FIG. 7).

In contrast the Iopamidol microparticles produced by spray-drying a suspension were mishappen and appeared crystalline in appearance. They also appeared somewhat smaller than the microparticles produced from a solution (see FIG. 8).

1. A method for producing microparticles of a particle-forming material, which method comprises the steps of
a) forming a suspension of a particle-forming material; and
b) spray-drying said suspension to form microparticles of the particle-forming material.

2. A method as claimed in claim 1, wherein step a), the formation of a suspension of the particle-forming material, is carried out by first dissolving the particle-forming material in a solvent, and then adding to the solution so formed a non-solvent for the particle-forming material, so as to bring about precipitation of the particle-forming material.

3. A method as claimed in claim 2, wherein the volume of non-solvent added to the solvent is greater than the volume of the solution of the particle-forming material in the solvent.

4. A method as claimed in claim 3, wherein the solvent/non-solvent mixture that is spray-dried in step b) comprises in excess of 60% v/v of non-solvent.

5. A method as claimed in claim 2, wherein the solvent is water.

6. A method as claimed in claim 2, wherein the non-solvent is ethanol.

7. A method as claimed in claim 2, wherein the solvent is an organic solvent.

8. A method as claimed in claim 1, wherein said spray-drying in step b) is carried out by spraying the suspension into a chamber containing a heated gas.

9. A method as claimed in claim 1, further comprising:
subjecting the suspension to homogenization prior to said spray-drying in step b.

10. A method as claimed in claim 1, wherein the particle-forming material is proteinaceous.

11. A method as claimed in claim 10, wherein the proteinaceous material is albumin.

12. A method as claimed in claim 11, wherein the albumin is human serum albumin.

13. A method as claimed in claim 10, wherein step a) is carried out by addition to a solution of the particle-forming material of a non-solvent for the particle-forming material, at a pH which is removed from the isoelectric point.

14. A method as claimed in claim 1, wherein the suspension contains from 0.1 to 50% w/v of particle-forming material.

15. A method as claimed in claim 1, wherein the suspension contains from 1 to 20% w/v of particle-forming material.

16. A method as claimed in claim 1, wherein the suspension contains from 2 to 10% w/v of particle-forming material.

17. A method as claimed in claim 1, wherein the particle-forming material is a therapeutically active agent.

18. A method as claimed in claim 1, wherein the particle-forming material is a pharmaceutical excipient.

19. A method as claimed in claim 18, wherein the pharmaceutical excipient is cholesterol.

20. A method as claimed in claim 1, wherein the particle-forming material is an imaging contrast enhancing agent.

21. A method as claimed in claim 20, wherein the imaging contrast enhancing agent is an X-ray imaging contrast agent.

22. A method as claimed in claim 21, wherein the contrast agent is Iopamidol.
